(19) United States
(12) Patent Application Publication
(10) Pub. No.: US 2005/0038372 A1
(43) Pub. Date: Feb. 17, 2005

(54) Applicator with a locking mechanism comprising a plurality of slits

(75) Inventors: Robert Clark Avery JR., Deerfield Township, OH (US); Wayne Grant Leslie, Loagan Township, IN (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/639,398

(22) Filed: Aug. 12, 2003

(51) Int. Cl. A61F 13/20

(52) U.S. Cl. 604/11

(57) ABSTRACT

An applicator device capable of housing an insertable element and an insertion member having an insertion end and a gripper end. The applicator includes a plunger that has a diameter, an insertion end and a gripper end. The insertion end of the plunger consists of a locking mechanism having a diameter greater than the diameter of the plunger. The locking mechanism also includes a plurality of slits.

### FIELD OF THE INVENTION

The present invention relates to an applicator capable of housing an insertable element having an improved locking mechanism region to improve the consumer's insertion experience.

### BACKGROUND OF THE INVENTION

As known in the art, applicators are used to both house materials intended to be inserted in a body cavity, such as a tampon or medicaments and to expel the material into the intended orifice. Typically applicators comprise an insertion member and a plunger. The material to be expelled from the applicator, such as an absorbent tampon, is positioned within the insertion member. The insertion member has a first end for insertion of the tampon and a second end for receipt of the plunger. To use the applicator, the consumer will position the first end appropriately, grasp the insertion member, and move the plunger in the insertion member towards the first end to insert the tampon. Some applicators also include a rim or stop to allow the user's to determine when the contents of the applicator have been fully expelled.

Various locking mechanism configurations have been proposed to facilitate the handling of the applicator and to improve the insertion experience. One approach is a tampon applicator as disclosed in U.S. Pat. No. 6,019,744, with a locking mechanism comprising of has a tongue shaped retention element that is integrally connected to the outer sleeve so as to be bendable and project through the longitudinal slit of the inner sleeve. Another approach is a tampon applicator as disclosed in U.S. Pat. No. 6,450,986, comprising a plunger having a rim comprising a shoulder projecting radialy outward from the plunger and a return margin. The rim creates an interference with the barrel of the applicator to provide a stop.

While these designs have worked well for plastic applicators, there remains a need for a less expensive tampon applicator with improved locking mechanisms. It is believed that the superior design and method of the present invention will provide an applicator with an improved locking mechanism comprised of paper, paperboard, cardboard or a combination thereof that will improve the consumer's insertion experience.

### SUMMARY OF THE INVENTION

The present invention comprises an applicator device capable of housing an insertable element and an insertion member having an insertion end and a gripper end. The applicator comprises a plunger having a diameter, an insertion end and a gripper end. The insertion end of the plunger comprises a locking mechanism having a diameter greater than the diameter of said plunger. The locking mechanism comprises a plurality of slits. In some embodiments, the locking mechanism is arranged so that the plunger is slideably introduced into the insertion member.

### BRIEF DESCRIPTION OF THE DRAWINGS

FIG. 1 is a side view of the applicator of the present invention.

FIG. 2 is a side view of the plunger of the present invention.

### DETAILED DESCRIPTION OF THE INVENTION

As used herein “applicator” refers to a device or implement that facilitates the insertion of a tampon, medicament, treatment device, visualization aid, or other into an external orifice of a mammal, such as the vagina, rectum, ear canal, nasal canal, or throat. Nonlimiting specific examples of such include any known hygienically designed applicator that is capable of receiving a tampon may be used for insertion of a tampon, including the so-called telescoping, tube and plunger and the compact applicators, an applicator for providing mediament to an area for prophylaxis or treatment of disease, a spectroscope containing a microcamera in the tip connected via fiber optics, a speculum of any design, a tongue depressor, a tube for examining the ear canal, a narrow hollow pipe for guiding surgical instruments, and the like.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

As used herein the term “tampon,” refers to any type of absorbent structure that is inserted into the vaginal canal or other body cavities for the absorption of fluid therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as rectangular, triangular, trapezoidal, semi-circular, hourglass, serpentine, or other suitable shapes. Tampons have an insertion end, withdrawal end, a length, a width, a longitudinal axis and a radial axis. The tampon's length can be measured from the insertion end to the withdrawal end along the longitudinal axis. A typical compressed tampon for human use is 30-60 mm in length. A tampon may be straight or non-linear in shape, such as curved along the longitudinal axis. A typical compressed tampon is 8-20 mm wide. The width of a tampon, unless otherwise stated in the specification, corresponds to the length across the largest cylindrical cross-section, along the length of the tampon.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

As used herein, “cm” is centimeters, and “mm” is millimeters.

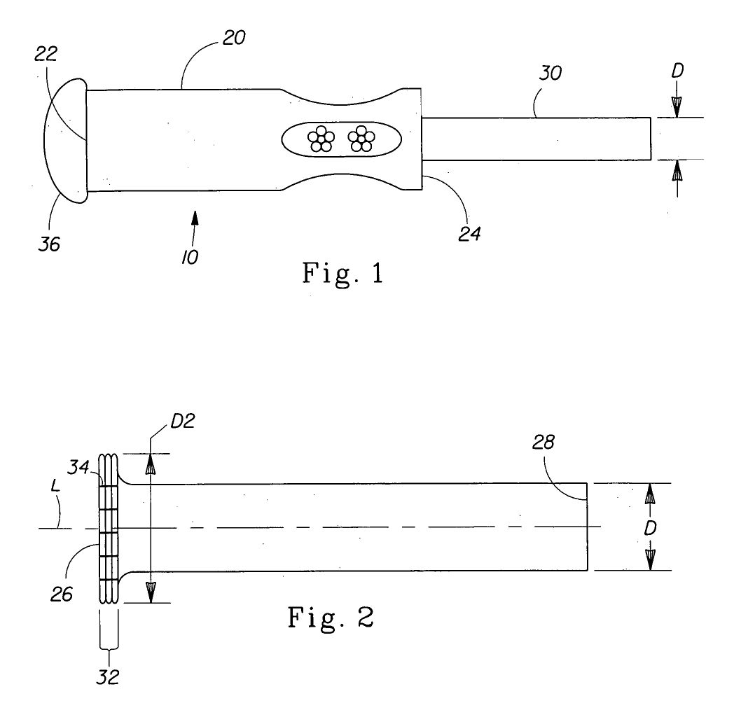

FIGS. 1 and 2 show the applicator 10 of the present invention. The applicator 10 includes an insertion member 20 adapted to house an insertable element, such a tampon 36 and a plunger 30. The insertion member 20 has an insertion end 22 and a gripper end 24. The applicator 10 comprises a plunger 30 having a diameter D, a longitudinal axis L, an insertion end 26 and a gripper end 28. The insertion end of the plunger 26 comprises a locking mechanism 32. The locking mechanism 32 comprises a plurality of slits 34. The locking mechanism 32 has a having a diameter D₂ greater than the diameter D of the plunger 30. In some embodiments, slits 34 are parallel to said longitudinal axis L.

The insertion member 20 is in the form of a spirally wound, convolutely wound or longitudinally seamed hollow tube, which is formed from paper, paperboard, cardboard or a combination thereof. The insertion member 20 may also be injection molded or formed from flexible plastic, such as thermoformed from plastic sheet or folded or wound from plastic film. The insertion member 20 may also be formed from a combination of paper and plastic. However, the plunger 30 is only formed from paper, paperboard, cardboard or a combination thereof. The plunger 30 is in the form of a spirally wound, convolutely wound or longitudinally seamed hollow tube.

The insertion member 20, also commonly referred to as an outer tube, has a relatively small diameter of about 10 millimeters to about 20 millimeters. The insertion member 20 has a wall with a predetermined thickness of about 0.1 millimeters to about 0.7 millimeter. The plunger 30 has a smaller diameter relative to the insertion member 20. The walls of the insertion member 20 and the plunger 30 can be constructed from a single ply of material or be formed from two or more plies that are bonded together to form a laminate.

The use of two or more plies or layers is preferred for it enables the manufacturer to use certain materials in the various layers that can enhance the performance of the tampon applicator 10. When two or more plies are utilized, all the plies can be spirally wound, convolutely wound or longitudinally seamed to form an elongated cylinder. For example, in some embodiments the wall can be constructed using a smooth thin ply of material on the outside or exterior surface that surrounds a coarser and possibly thicker ply. In the embodiments that contain at least three plies, the middle ply can be the thicker ply and the interior and exterior plies can be smooth and/or slippery to facilitate expulsion of the tampon 36 and to facilitate insertion of the insertion member 20 into a woman's vagina, respectively. By sandwiching a thick, coarser ply of material between two thin, smooth plies, an inexpensive insertion member 20 can be provided which is very functional. The wall may contain one to four plies, although more plies can be utilized if desired. As well, the ends of the insertion member 20 can be lipped.

An adhesive, such as glue, or by heat, pressure, ultrasonics, etc, can hold the plies forming the wall together. The adhesive can be either water-soluble or water-insoluble. A water-soluble adhesive is for environmental reasons in that the wall will quickly break apart when it is immersed in water. Such immersion will occur should the insertion member 20 be disposed of by flushing it down a toilet. Exposure of the insertion member 20 to a municipal's waste treatment plant wherein soaking in water, interaction with chemicals and agitation all occur, will cause the wall to break apart and evenly disperse in a relatively short period of time.

The inside diameter of the insertion member 20 is usually less than about 0.75 inches (about 19 mm) and preferably less than about 0.625 inches (about 16 mm). Although the exterior diameter of tampons 36 does vary, most tampons 36 utilized by women have an external diameter of less than about 0.75 inches (about 19 mm). However, if one desired to use this invention to administer medication to an animal, such as a farm animal or other mammal, larger size tampons 36, which would require insertion members with a larger diameter, could be used.

Alternatively, the material can be overlapped into a tubular configuration. Spirally or convolutely winding the insertion member 20 into a cylindrical tube is especially advantageous when the insertion member 20 is formed from a laminate. In the case of other tube construction methods such as fiber or plastic molding, or integral tube forming (e.g. thermoforming plastic) no seams will be present and the corrugations could optionally be formed as part of the tube molding or forming process.

The insertion member 20 is sized and configured to house an insertable element, such as an absorbent tampon 36. As stated above, the insertion member 20 should have a substantially smooth exterior surface that will facilitate insertion of the insertion member 20 into a woman's vagina. When the exterior surface is smooth and/or slippery, the insertion member 20 will easily slide into a woman's vagina without subjecting the internal tissues of the vagina to abrasion. The insertion member 20 can be coated to give it a high slip characteristic. Wax, polyethylene, a combination of wax and polyethylene, cellophane, clay, mica and other lubricants are representative coatings that can be applied to the insertion member 20 to facilitate comfortable insertion.

The applicator 10 of the present invention can have geometries or cross-sections that are useful to contain the object to be inserted. Often, the shape of the tampon 36 contained suggests the shape of the insertion member 20, but departures from this general rule may be made such that a cylindrical tampon 36 may be house in a rectangular shaped applicator, for example. Thus, the insertion member 20 may take on numerous cross-sectional shapes including without limitations, circular, oval, polygonal (e.g. trapezoidal, rectangular, triangular) and the like. In addition the insertion member 20 can be substantially elongated, curved or flexible, or it can take on other shapes that are apparent to one of ordinary skill in the art.

The plunger 30 is slideably configured within the inside diameter of the insertion member 20. The locking mechanism 32 of the plunger 30 is designed to provide a stop to allow users to determine when the contents of the applicator 10 are fully expelled. The locking mechanism 32 also insures that the plunger 30 remains slideably configured inside the insertion member 20 so that the plunger 30 may not be pulled from the insertion member 20 during insertion or during shipment. While specific tube geometry is not critical, in order for the locking mechanism 32 to prevent separation of the insertion member 20 and plunger 30, an interference with at least a part of the insertion member 20 must be created. This can be accomplished by configuring the locking mechanism 32 to have a having a diameter D₂ greater than the diameter D of the plunger 30. The locking mechanism 32 is configured such that it projects radially outward by at least about 0.1 mm to at least about 3 mm from the plunger 30. The locking mechanism 32 can have from about 2 to about 24 slits 34. The locking mechanism 32 can have from about 2 to about 12 slits 34 or from about 3 to about 6 slits 34. The plunger 30 is inserted into the insertion member 20 such that the locking mechanism 32 of the plunger 30 is slideably introduced into the insertion member 20.

The applicator of the present invention is made by first providing an insertion member 20 having an insertion end 22 and a gripper end 24. Next, a plunger 30 is provided having a diameter, an insertion end 26, and a gripper end 28. A plurality of slits 34 are produced on the insertion end 26 of the plunger 30. A plurality of individual knives, shears, laser, and a rotating slitting wheel may be used to slit of the insertion end 26 of the plunger 30. In some embodiments, the slits 34 may be produced by a pushing the insertion end 26 of the plunger 30 into a plurality of knives arranged in a pattern. After the insertion end 26 of the plunger 30 is slit, the insertion end 26 of the plunger 30 is deformed to form a locking mechanism. The deformation of the insertion end 26 of the plunger 30 is accomplished by subjecting the insertion end 26 of the plunger 30 to compression. In some embodiments, the deformation of the insertion end 26 of the plunger 30 may be accomplished by compressing the insertion end 26 of the plunger 30 between a male and female die set. The deformation of the insertion end 26 of the plunger 30 may be also be accomplished by compressing the plunger 30 along the longitudinal axis between converging plates or belts. Impacting the plunger against the surface of fixed member may also deform the insertion end 26 of the plunger 30. Holding the plunger while impacting it with the surface of moving member may also deform the insertion end 26 of the plunger 30. Next, the plunger is inserted into the insertion member 20. In some embodiments, the locking mechanism 32 is arranged for the plunger 30 to be slideably introduced into the insertion member 20.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An applicator device capable of housing an insertable element comprising:
an insertion member having an insertion end and a gripper end;
a plunger having a diameter, an insertion end and a gripper end; said insertion end of said plunger comprising a locking mechanism having a diameter greater than said diameter of said plunger; said locking mechanism comprising a plurality of slits.

2. The applicator according to claim 1 wherein said plunger is comprised of paper.

3. The applicator according to claim 1 wherein said insertion member is comprised of paper.

4. The applicator according to claim 1 wherein said plunger has a longitudinal axis.

5. The applicator according to claim 1 wherein said slits are parallel to said longitudinal axis.

6. The applicator according to claim 1 wherein said locking mechanism comprising from about 2 to about 24 slits.

7. The applicator according to claim 1 wherein said locking mechanism comprising from about 2 to about 12 slits.

8. The applicator according to claim 1 wherein said locking mechanism comprising from about 3 to about 6 slits.

9. The applicator according to claim 1 wherein the locking mechanism of said plunger is slideably introduced into said insertion member.
